(19) United States
(12) Patent Application Publication
(10) Pub. No.: US 2021/0220560 A1
(43) Pub. Date: Jul. 22, 2021

(54) Injection Device and Container for an Injection Device

(71) Applicant: SANOFI, Paris (FR)

(72) Inventor: Michael Helmer, Frankfurt am Main (DE)

(21) Appl. No.: 17/054,381

(22) PCT Filed: May 15, 2019
(86) PCT No.: PCT/EP2019/062428

§ 371 Date: Nov. 10, 2020

(30) Foreign Application Priority Data
May 17, 2018 (EP) 18305614.2

(51) Int. Cl. A61M 5/24; A61M 5/315; A61M 5/20; A61M 5/32

(52) U.S. Cl. CPC A61M 5/2422; A61M 5/31515; A61M 5/20; A61M 2005/3231; A61M 2005/206; A61M 2005/2407; A61M 2005/323; A61M 5/3221

(57) ABSTRACT

In one aspect the present disclosure relates to an injection device. The injection device includes a housing configured to receive a container; a drive mechanism having a plunger configured to urge against a bung in the container in a distal direction to expel the dose of the medicament from the container, wherein the plunger is configured to releasably connect to the bung to form a plunger bung assembly; a container moving mechanism configured to move the container relative to the housing between a proximal replacement position and a distal injection position; at least one retainer configured to prevent a proximally directed movement of the container relative to the housing. The drive mechanism is configured to retract the plunger and the plunger bung assembly along the proximal direction relative to the tubular barrel of the container.

### CROSS REFERENCE TO RELATED APPLICATIONS

The present application is the national stage entry of International Patent Application No. PCT/EP2019/062428, filed on May 15, 2019, and claims priority to Application No. EP 18305614.2, filed on May 17, 2018, the disclosures of which are incorporated herein by reference.

### TECHNICAL FIELD

The present disclosure relates to the field of injection devices and containers for such injection devices, wherein the container is at least partially filled with an injectable medicament. In one aspect the disclosure relates to so-called auto injectors comprising a housing in which the container and/or an injection needle connected to the container is or are displaceable relative to the housing. According to a further aspect the disclosure relates to reusable auto injectors, wherein a medicament container or a cartridge containing the medicament can be replaced.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors have to meet a number of user-specific requirements. For instance, with patient's suffering chronic diseases, such like diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

Typically, such devices comprise a housing or a particular cartridge holder, adapted to receive a medicament container, e.g. in form of a cartridge at least partially filled with the medicament to be expelled. The device further comprises a drive mechanism, usually having a displaceable plunger or piston rod to operably engage with a bung or piston of the medicament container or cartridge. By means of the drive mechanism and its piston rod, the bung or piston of the cartridge is displaceable in a distal direction or dispensing direction and may therefore expel a predefined amount of the medicament via a piercing assembly, e.g. in form of an injection needle, connected to or is releasably connectable with an outlet end of the medicament container. With reusable drug delivery devices an empty cartridge is replaceable by a filled one. In contrast to that, drug delivery devices of disposable type are to be entirely discarded when the medicament in the cartridge has been dispensed or used-up.

With some injection devices, such as auto injectors there may evolve a certain injury risk from an exposed tipped end of an injection needle. When the injection device is configured or designed as a reusable injection device a medicament container or cartridge filled with the medicament has to be replaced after a certain time or after the content of the medicament container has been expelled or dispensed. Especially during the process of replacing of a medicament container the tipped end of an injection needle may entail or may represent a certain risk of injury.

With some injection devices the injection needle is non-detachably connected to the medicament container. With such injection devices the replacement of the medicament container with the injection needle attached thereto might be rather cumbersome and may impose an undesired burden to the user of the device when replacing the medicament container. Even though users of such devices may be encouraged, even instructed to return a safety cap back onto the needle before replacing the medicament container, there still remains a certain risk of injury.

### SUMMARY

In some aspects, the present disclosure provides an improved injection device configured for injecting of a dose of a liquid medicament which device provides and enables a rather easy, user friendly and non-hazardous replacement of a medicament container. In other aspects, the present disclosure provides a container for a liquid medicament configured for operation with such an improved injection device. The interaction of the container and the injection device can provide improved patient safety and can provide a reduced risk of injury that may otherwise arise from an exposed needle tip.

In other aspects, the present disclosure provides a method of exchanging a medicament container in an injection device allowing and supporting a rather easy, straight forward and failure safe replacement of a medicament container in the injection device providing a high degree of patient safety.

In one aspect there is provided an injection device for injecting of a dose of a liquid medicament. The injection device comprises a housing configured to receive a container. The container is a medicament container and comprises a tubular barrel filled with a liquid medicament. The container is sealed by a bung or piston in a proximal direction or at a proximal end. The bung is slidably arranged inside the barrel. The injection device further comprises a drive mechanism having a plunger that is configured to urge against the bung of the container in a distal direction. The drive mechanism and the plunger are configured to advance the plunger in a dispensing direction, i.e. towards a dispensing or expelling end of the injection device, typically in distal direction. The plunger is configured to exert a dispensing pressure onto the bung so as to move the bung relative to the barrel, thus expelling a well-defined amount, i.e. a dose, of the medicament through a distal outlet of the medicament container.

The plunger is configured to releasably connect to the bung to form a plunger bung assembly. Once a plunger bung assembly has been established or formed, namely when the plunger is connected to the bung of the cartridge, the plunger and the bung are bi-directionally connected. This means, a distally and a proximally directed motion of the plunger relative to the container equally transfers into a distally and a proximally directed displacement of the bung relative to the barrel of the container, respectively. When the plunger bung assembly is established the bung is pushable by the plunger in distal direction and the bung is also retractable by the plunger in the opposite proximal direction. The releasable connection of bung and plunger enables a well-defined detaching of the bung from the plunger to enable a replacement of, e.g. an empty medicament container.

The injection device further comprises a container moving mechanism that is configured to move the container relative to the housing of the injection device between a proximal replacement position and a distal injection position. Hence the medicament container is typically slidably displaceable inside the housing of the injection device. When in the proximal replacement position the medicament container and an optional injection needle attached thereto is or are in an undeployed configuration. When in the distal injection position the container and/or the injection needle is or are in a deployed configuration. In the distal injection position the injection needle may project or protrude from a dispensing end of the housing of the injection device.

Typically, the container moving mechanism is activatable after the housing of the injection device has been brought in contact with a pierceable skin portion. Upon deploying or activating of the container moving mechanism the medicament container and the injection needle advance in distal direction to arrive at the distal injection position. Upon arriving at the distal injection position or prior to an arrival in the distal injection position the injection needle pierces or penetrates the skin portion.

The injection device further comprises at least one retainer that is configured to prevent a proximally directed movement of the container relative to the housing. The retainer may be configured to prevent a proximally directed movement of the container when in one of the proximal replacement position and the distal injection position. The drive mechanism of the injection device is further configured to retract the plunger and the plunger bung assembly along the proximal direction relative to the tubular barrel of the container. The drive mechanism is hence configured to retract the plunger bung assembly towards the proximal direction relative to the tubular barrel while the medicament container is retained in one of the proximal replacement position and the distal injection position by the at least one retainer.

Alternatively, it is also conceivable to achieve a retraction of the plunger relative to the tubular barrel towards the proximal end of the tubular barrel by retaining and immobilizing the plunger and the plunger bung assembly while displacing or moving the tubular barrel towards the distal direction. Here, the container moving mechanism may be configured to displace and to move the container in distal direction while the plunger bung assembly is fixed relative to the housing of the injection device. In this way a likewise movement of the plunger bung assembly towards the proximal direction relative to the tubular barrel of the container can be obtained.

The injection device is particularly configured to cooperate with a particular type of medicament containers. Such medicament containers comprise a tubular-shaped barrel and a bung sealing a proximal end of the barrel, thus confining a filling volume of the barrel and hence of the medicament container towards the proximal direction. The bung typically comprises a counterpart connector configured to releasably engage with the connector of the plunger of the injection device so as to form the plunger bung assembly. The counterpart connector of the bung typically faces towards the plunger, i.e. towards a proximal end of the container. The bung further comprises a connector facing towards a distal end of the barrel.

The container further comprises a needle holder that is configured to hold an injection needle. The needle holder is retractable inside the tubular barrel. Typically the needle holder is arranged at a distal end of the medicament container. It is retractable into the interior of the barrel in proximal direction relative to the barrel. Optionally, the needle holder comprises a seal in fluid tight engagement with the barrel. The needle holder is displaceable relative to the barrel inside the barrel. With the container at least one of the needle holder and the injection needle comprises a counterpart connector configured to engage with the connector of the bung so as to form a mechanical interconnection with the bung. In this way and when arriving at a distal most position after expelling the entire content of the medicament from the medicament container the connector of the bung releasably or non-detachably connects with the counterpart connector of the needle holder. By means of the counterpart connector of the bung, the bung itself is connectable to the plunger of the drive mechanism of the injection device. Now and since the needle holder is connected to the bung and since the bung is connected to the plunger, the drive mechanism of the injection device is operable to move the plunger towards a proximal direction relative to the barrel of the container thus displacing the plunger bung assembly together with the needle holder in the proximal direction relative to the barrel of the cartridge.

In effect, the needle attached to or connected to the needle holder is subject to a proximally directed displacement relative to the barrel of the container thus retracting the tipped distal needle end into the interior of the barrel to such an extent, that a distal end of the injection needle is located proximally from the distal end of the barrel of the container. In this way, the barrel of the container serves as a needle shield or needle guard after the content of the container has been expelled. Insofar, the drive mechanism configured to retract the plunger and the plunger bung assembly is also configured to retract a needle holder attached to the bung so as to retract an injection needle inside the barrel of the medicament container. Thereafter, the medicament container is replaceable by another one. Since the tipped end of the injection needle is located inside the barrel a danger of injury can be substantially reduced.

According to another example the at least one retainer comprises a distal retainer configured to retain the container in the distal injection position. The distal retainer may be located at or near an axial position that coincides with the proximal end of the medicament container when located in the distal injection position. The distal retainer may engage or may abut with a proximal end of the container so as to prevent a proximally directed displacement of the container relative to the housing when in the distal injection position. In this way a proximally directed retraction force provided by the drive mechanism and the plunger and hence acting on the plunger bung assembly leads to a respective proximally directed movement of the bung relative to the barrel of the medicament container.

The distal retainer is hence configured to fix the medicament container inside the housing at least with regard to a proximally directed movement of the container relative to the housing. While the medicament container is kept in the distal injection position the drive mechanism is configured to retract the plunger and the plunger bung assembly in proximal direction so as to induce a proximally directed displacement of the bung relative to the barrel of the medicament container. If the bung is further connected with a needle holder also the needle holder and the injection needle connected thereto is retracted inside the barrel of the medicament container.

The distal retainer does not have to be located near a proximal end of the medicament container when the latter is in the distal injection position. The distal retainer may also be located near or close to a distal end of the medicament container when in the distal injection position. The distal retainer may comprise or form an abutment for a correspondingly-shaped abutment section of the medicament container thus preventing a proximally directed displacement of the container relative to the housing and relative to the distal retainer.

In a further example the distal retainer is deactivatable to allow and to support a proximally directed movement of the container relative to the housing towards the proximal replacement position. The distal retainer is at least temporally deactivatable. When deactivated the container is allowed to move from the distal injection position to the proximal replacement position. The container may then pass by the distal retainer. In order to deactivate the distal retainer the abutment provided by the distal retainer may be pivotably connected to the housing.

For instance, the distal retainer may pivot with regard to a longitudinal or tangential axis with regard to the tubular shape of the barrel so as to protrude inwardly from a sidewall of the housing thus providing an axial abutment for the medicament container. In such a protruding configuration a proximally directed movement of the medicament container relative to the housing is prevented and blocked. By pivoting or moving the distal retainer out of the outer circumference of the medicament container the distal retainer gives way for a proximally directed movement of the medicament container relative to the housing. Deactivating, e.g. pivoting or moving of the distal retainer from an activated into a deactivated position may be controlled by an electronic control of the injection device. Alternatively, a movement of the distal retainer may also be governed by an all mechanically implemented drive mechanism of the injection device. The drive mechanism with the plunger may be all mechanically implemented or may comprise an electromechanical drive controlled by an electronic control of the injection device.

In another example the at least one retainer comprises a proximal retainer configured to retain the container in the proximal replacement position. The proximal retainer is particularly configured to impede a proximally directed movement of the container when in the proximal replacement position. In other words the proximal retainer provides an end stop for the medicament container when arriving at the proximal replacement position. Any further proximally directed displacement of the container is thus impeded. The proximal retainer may provide the same functionality as the distal retainer. It is hence conceivable that after completion of a dose injection procedure the container moving mechanism in a first step moves the medicament container from the distal injection position towards and into the proximal replacement position. Thereafter, a further proximally directed movement of the medicament container is blocked and impeded by the proximal retainer. In a second step, the drive mechanism retracts the plunger and the plunger bung assembly further in proximal direction so as to move the bung of the medicament container in proximal direction relative to the barrel of the medicament container. Typically, the needle holder connected to the bung is likewise retracted into the barrel of the medicament container.

It is generally sufficient when the injection device comprises only one retainer, namely the distal retainer or the proximal retainer. If there is provided only a distal retainer a retraction of the bung and of the needle holder into the barrel of the medicament container is conducted while the container is in the distal injection position. If the injection device is equipped only with the proximal retainer the container may be initially moved from the distal injection position into the proximal replacement position. A retraction of the bung and the needle connected thereto into the barrel of the medicament container is then conducted when the container is located in the proximal replacement position.

Disconnecting of the plunger from the bung may be supported by the proximal retainer. A mutual interconnection between the plunger and the bung may be of load limiting type. The interconnection remains intact as long as a force or load to be transferred across the interface of the plunger and the bung is below a predefined maximum load. As the maximum load is exceeded, the interconnection is abrogated. Typically, friction forces between the barrel, the plunger, the bung and the needle holder are substantially smaller than the predefined maximum load across the plunger bung interconnection. In this way and as long as the bung is subject to a proximally directed displacement relative to the barrel the interconnection between the plunger and the bung remains intact.

According to a further example the proximal retainer does not only serve to impede a proximal displacement of the medicament container beyond the proximal replacement position but also serves to prevent a withdrawal of the bung from the barrel of the medicament container. Insofar, the proximal retainer may provide an axial abutment for both, the medicament container and for the bung slidably displaced therein. In a typical example the medicament container comprises an open ended proximal end. When in the proximal replacement position the proximal end of the medicament container may be in abutment with the proximal retainer that is fixed or integrally formed with the housing of the injection device.

The proximal retainer may slightly protrude radially inwardly from the sidewall of the container. In this way, the proximal container also provides a longitudinal or axial abutment for the bung of the medicament container. Here, the bung cannot be detached from the barrel of the medicament container. Moreover, when the bung is in abutment with the proximal retainer any further proximally directed force effect exerted by the plunger leads to an increase of the load across the plunger bung interface until the predefined maximum load or a load larger than the predefined maximum load is present to the interface.

Accordingly, the interconnection of the bung and the plunger may abrogate. Insofar, the proximal retainer provides and supports a force-induced detachment of the plunger and the bung. Typically, the plunger and the bung may form or comprise a snap fit interconnect which is established by urging the plunger towards and against a proximal end of the bung. Typically, a force required to establish or to form the snap fit connection between the plunger and the bung is smaller than or equal to a friction force required to move the bung relative to the sidewall of the barrel. A force required for detaching the interconnect between the plunger and the bung is typically much larger than the sum of friction forces for displacing the bung in proximal direction relative to the cartridge, for displacing the needle holder relative to the barrel in proximal direction and for moving the container from the distal injection position towards and into the proximal replacement position.

According to a further example the plunger comprises a connector to releasably connect with a counterpart connector of the bung. One of the connector and the counterpart connector typically comprises a female connecting structure and the other one of the connector and the counterpart connector comprises a correspondingly-shaped male connecting structure. The connector and the counterpart connector are further configured to establish a positive fit or positive locking between the plunger and the bung. As mentioned above, the mutual connection of the plunger and the bung may be established simply by inserting a male locking structure into a correspondingly-shaped female locking structure. The force for establishing of the interconnect between the plunger and the bung may be substantially smaller than a force required to disconnect the connector and the counterpart connector.

Typically, the interconnection is of load limiting type. It will remain intact as long as a drag force applied by the plunger onto the bung in proximal direction is below a predefined maximum load. If the predefined maximum load is exceeded the connector and the counterpart connector are configured to disassemble in order to provide a well-defined abrogation or disconnection of the plunger and the bung. This allows and supports a mechanical decoupling of the medicament container from the drive mechanism and hence from the injection device. After the connector and the counterpart connector have been disconnected the medicament container can be removed and eventually replaced by another medicament container. The connector and the counterpart connector may be configured as mechanical connectors.

The connector and the counterpart connector may form a snap fit interconnection or a bayonet type interconnection. With a bayonet interconnection for connecting the connector and the counterpart connector at least a certain rotation of one of the connector and the counterpart connector is required relative to the other one of the connector and the counterpart connector. Such a rotation may be induced or conducted by the plunger of the drive mechanism. The interconnection between the plunger and the bung comprises a well-defined degree of tensile stiffness. Up to a certain and predefined maximum load the connection remains intact. When the predefined maximum load or a load larger than the predefined maximum load is applied across the interface of the connector and the counterpart connector the mutual interconnection is configured to abrogate thus disconnecting the plunger from the bung.

Alternative to a mechanical interconnection it is also conceivable that the connector and the counterpart connector comprise mutually corresponding magnetic interconnectors.

According to a further example the container moving mechanism is configured to slidably displace the container between the proximal replacement position and the distal injection position. The container moving mechanism comprises a drive to induce or to control a motion of the container relative to the housing. The drive may be implemented as an electromechanical drive or may comprise a purely mechanically implemented drive that may be driven by a source of mechanical energy, such as a biased spring. The drive may comprises both, an electromechanical drive and a spring in order to induce a distally and a proximally directed motion of the medicament container relative to the housing.

For instance, an electromechanical drive may be configured to move the container from the proximal replacement position towards and into the distal injection position against the action of a spring element. In this way, a return motion of the container, hence from the distal injection position towards and into the proximal replacement position may be at least supported or entirely governed by the spring. It is also conceivable, that the electromechanical drive is configured to induce both, a distally directed motion and a proximally directed motion of the container relative to the housing so as to move the container between the proximal replacement position and the distal injection position.

The drive for displacing the medicament container relative to the housing may be provided in addition to the drive mechanism, wherein the drive mechanism is configured to interact with the bung of the medicament container. The drive for displacing the medicament container may comprise an electromechanical drive and a gearbox mechanically connected to a sliding mechanism that is configured to receive the container. The sliding mechanism may be operated by the drive so as to displace the container relative to the housing.

According to a further example the drive for operating the container moving mechanism is provided by the plunger when connected to the bung and when the plunger is moved relative to the housing. In this way, a separate drive for moving of the medicament container between the proximal replacement position and the distal injection position becomes superfluous. The drive mechanism and the plunger may thus provide a twofold function, namely to move the container between the proximal replacement position and the distal injection position and to move the bung relative to the barrel of the container. Integrating of a drive of the container moving mechanism into the drive mechanism is therefore beneficial in terms of reducing the package size as well as for reducing the manufacturing and assembly costs for the injection device.

In another example the injection device is equipped with the container arranged inside the housing. The container is typically pre-filled with the liquid medicament. The container may comprise a pre-filled syringe or a pre-filled cartridge. The pre-filled syringe may be equipped with an injection needle when arranged inside the housing of the injection device. The injection needle may be non-detachably connected to the container and may be covered by a protective cap that is to be removed prior to conduct an injection procedure.

With another example and when the container is configured as a cartridge the cartridge comprises a seal at a distal end that is to be pierced by a double-tipped piercing assembly, e.g. in form of a needle assembly. For this, the distal end of the medicament container or a distal end of the injection device comprises a fastener for a detachable needle assembly. The needle assembly, typically comprising a cupped receptacle and a double-tipped injection needle may have to be individually attached to the outlet of the medicament container thus penetrating the pierceable seal of the container. In this way the injection needle gets in fluid communication with the inner volume of the medicament container. Also here, it may be of particular benefit when the needle assembly attached to the container can be retracted into the barrel of the container after the content of the container has been expelled.

According to a further example the container comprises a needle holder configured to hold an injection needle. The needle holder is retractable inside the tubular barrel. At least a portion of the needle holder is permanently arranged inside the tubular barrel. Typically, the portion of the needle holder located inside the barrel serves as a seal or forms a seal in fluid tight engagement with an inside of the barrel. The needle holder is configured for attachment of a needle assembly thereto. Alternatively, the needle holder is readily equipped with an injection needle extending through the needle holder in longitudinal direction. When the medicament container is implemented as a pre-filled syringe the injection needle is typically non-detachably fastened or connected to the needle holder. When implemented as a cartridge the needle holder may be configured for releasable connection with a needle assembly. For this the needle holder may comprise a fastener protruding distally from the distal end of the cartridge. The fastener of the needle holder is configured to engage with a correspondingly-shaped counter fastener of a needle assembly. The fastener and the counter fastener of the needle holder and of the needle assembly may be threadedly engaged or may form a snap fit connection.

According to a further example the bung of the medicament container comprises a connector facing towards the needle holder. At least one of the needle holder and the injection needle comprises a counterpart connector facing towards the bung. The connector and the counterpart connector are configured to form a mechanical interconnection between the bung and the needle holder. In this way and as the bung is displaced by the plunger into abutment with the needle holder, the connector and the counterpart connector mutually engage so as to establish a mechanical connection between the bung and the needle holder.

Since the bung is connected to the plunger and since the plunger is retractable in proximal direction relative to the barrel of the container also the bung connected to the plunger and the needle holder connected to the bung become subject to a respective proximally directed motion relative to the barrel of the container. In effect, the needle holder and the injection needle are subject to a retracting motion into the barrel of the medicament container. A danger of injury when subsequently exchanging the empty medicament container can be thus reduced. For the user or patient there is no longer a need to return a protective cap onto the exposed needle or to detach the injection needle after the last dose has been expelled from the medicament container.

In a further example the needle holder comprises a seal in fluid tight engagement with the barrel of the container. In this way, the needle holder fulfills a double function. It serves to support a needle at a distal end of the tubular-shaped barrel of the container. In a second aspect the needle holder also seals the distal end of the barrel of the medicament container. The seal may comprise a polymeric or elastomeric material, such as natural or synthetic rubber providing a fluid tight sealing engagement with the inside of the barrel of the container. The seal may be of similar shape and/or similar material compared to the bung of the medicament container. Typically, the needle holder and at least the seal thereof comprises an outer shape and circumference that matches with an inside shape of the sidewall of the barrel of the container. In this way, the seal and the needle holder can be retracted in proximal direction relative to the barrel of the medicament container.

For inducing a motion of the needle holder and the bung in proximal direction relative to the barrel of the medicament container the plunger bung assembly and the needle holder may be fixed relative to the housing. The medicament container may then be moved towards the distal direction. In this way, the bung and the needle holder experience a proximally directed movement relative to the barrel of the medicament container. After completion of an injection procedure this may be accomplished by moving the medicament container from the distal injection position into the proximal replacement position without inducing a relative motion between the plunger and the barrel of the medicament container. Thereafter and in a second step the plunger, the bung and optionally also the needle holder connected to the bung can be immobilized by means of the drive mechanism of the injection device when a separate drive of the container moving mechanism may be activated so as to move the barrel of the container in distal direction, e.g. towards or into the distal injection position, which due to the axially fixed plunger bung assembly leads to an effective retraction of the bung and of the needle holder into the barrel of the medicament container.

According to another aspect the disclosure relates to a container for a liquid medicament. The container comprises a tubular-shaped barrel and a bung sealing a proximal end of the barrel. The bung comprises a connector facing towards a distal end of the barrel. The container further comprises a needle holder that is configured to hold an injection needle. The needle holder is retractable inside the tubular barrel. The needle holder comprises a seal in fluid tight engagement with the barrel. Typically and in an initial configuration the needle holder is arranged at a distal end of the tubular-shaped barrel.

At least one of the needle holder and the injection needle comprises a counterpart connector configured to engage with the connector to form a mechanical interconnection with the bung. The counterpart connector typically faces towards the proximal end of the barrel and hence towards the bung. The counterpart connector may be provided at a proximal end of the needle extending through the needle holder. Alternatively, the counterpart connector is integrated or arranged at the needle holder, typically at a proximal side or proximal end of the needle holder, whereas a distal end of the needle holder is configured for a releasable connection with a separate needle assembly.

The container is particularly adapted for use with an injection device as described above. The mutually corresponding connector and counterpart connector of the bung and one of the needle holder and the injection needle is beneficial to establish a mechanical connection between the bung and the needle holder. After expelling of the content of the medicament from the interior of the barrel and when the bung reaches a distal most axial position inside the barrel the mechanical connection between the bung and the needle holder is automatically established, e.g. by means of a snap fit connection provided by the connector and the counterpart connector of the bung and of one of the needle holder and the injection needle. Thereafter and when applying a proximally directed retraction force onto the bung, the bung also retracts the needle holder and the injection needle attached thereto into the interior of the barrel so that a distal end of the injection needle no longer protrudes from the barrel of the medicament container.

Typically and according to a further example the bung also comprises a counterpart connector facing towards the proximal end of the barrel. By means of the counterpart connector the bung is releasably connectable with the plunger of the injection device. Retracting of the plunger relative to the barrel then induces a corresponding proximally directed motion of the bung and of the needle holder relative to the barrel. The interconnection between the plunger of the drive mechanism, the bung and the needle holder is of particular benefit because the existing drive mechanism of the injection device can be used to retract the needle into the barrel. This allows and enables a rather simple, reliable and failure safe design of the medicament container.

According to another example the barrel may comprise a vitreous barrel or may be made of a plastic material. The barrel may comprise a radially inwardly extending flange at a distal end providing an axial abutment for the needle holder axially displaceably arranged inside the barrel. The needle holder or at least a portion thereof may protrude through the orifice formed by the radially inwardly extending flange at the distal end of the tubular-shaped sidewall of the barrel.

Optionally, also the proximal end of the barrel may be provided or equipped with a flange. The flange at the proximal end of the barrel may protrude radially outwardly in order to provide a well-defined axial abutment with at least one retainer of the injection device.

In another embodiment the needle holder comprises a socket configured to protrude axially through an orifice at a distal end of the barrel. The orifice may be surrounded or confined by a radially inwardly extending annular flange at the distal end of the tubular-shaped barrel. The socket protruding distally through the orifice comprises a fastener, e.g. in form of an outer thread to engage with a correspondingly-shaped counter fastener, e.g. an inner thread of a cup-shaped needle assembly. In this way, standard needles widely used with pen-type injectors can be used to releasably attach to the socket of the needle holder. Here, the needle holder, in particular the socket thereof comprises a pierceable closure at a distal end that is configured to be penetrated by a proximally directed tipped portion of the double-tipped injection needle of the needle assembly.

Typically, the needle holder comprises a radially widening shoulder portion axially adjacent to the socket. The circumference or the diameter of the shoulder portion is larger than the cross-section of the orifice. Typically, the diameter or cross-section of the shoulder portion matches the diameter or cross-section of the tubular-shaped sidewall of the barrel. In this way, the shoulder portion of the needle holder serves as a seal being in fluid tight engagement with the inside of the barrel.

According to another example an inner diameter of the orifice of the barrel of the container is larger than or equal to an outer diameter of the cup-shaped needle assembly. This allows and enables retraction of the needle holder together with the needle assembly into the barrel of the medicament container. In this way, the needle holder engaged with the cup-shaped needle assembly can be retracted into the interior of the barrel after the medicament has expelled from the interior of the barrel.

In another example the container further comprises a rotation lock configured to prevent a rotation between the needle holder and the barrel. The rotation lock comprises at least one radial protrusion configured to engage with at least one complementary shaped radial recess. Typically, the at least one protrusion is located on one of the needle holder and the barrel and the recess is located on the other one of the needle holder and the barrel.

With some examples the radial protrusion faces inwardly from an inwardly facing circumference of a flange confining an orifice of the barrel. The radial recess is then located on an outer surface of the needle holder, typically in an axial midsection of the needle holder extending in longitudinal direction through the orifice of the barrel. With other examples, the radial protrusion is provided on an outside surface of the needle holder and the complementary shaped recess is provided on a radially inwardly facing circumference of the flange of the orifice of the barrel.

With either configuration the rotation lock prevents and impedes a rotation of the needle holder relative to the barrel. This is of particular benefit when an injection needle has to attached to the needle holder by way of a screwing motion.

Typically, the at least one protrusion and the complementary shaped recess extend in longitudinal direction. For an optional disengagement of the rotation lock between the needle holder and the barrel the needle holder can be retracted in longitudinal direction into the interior of the barrel as described above. In this way, the rotational interlock of the recess and the protrusion can be abrogated.

According to another aspect the disclosure also relates to a method of exchanging a medicament container in an injection device. Typically, this method can be conducted with an injection device and with a container for such an injection device as described above. The method of exchanging of the medicament container starts after completion of an injection procedure. It comprises at least one of the following steps, namely retracting of a needle holder into a barrel of the medicament container or moving of the medicament container relative to a housing of the injection device from a distal injection position into or towards a proximal replacement position. When retracting the needle holder into the barrel the barrel may remain stationary relative to a housing of the injection device. It may remain at the distal injection position.

When moving the medicament container relative to the housing towards and into the proximal replacement position the needle holder may remain stationary relative to the barrel of the cartridge. With either method step the needle of the injection device is retracted in proximal direction and is hence withdrawn from the skin of a patient. It is even conceivable to conduct both of the above mentioned steps, namely to retract the needle holder into the barrel of the medicament and to move the medicament container relative to the housing in proximal direction, hence from the distal injection position into or towards the proximal replacement position. The needle holder may be retracted into the barrel before the medicament container is moved towards the proximal replacement position. Alternatively, the needle holder may be retracted into the barrel after the medicament container has been moved into or towards the proximal replacement position. The two method steps, namely retracting the needle holder and moving of the medicament container relative to the housing may also be conducted concurrently or simultaneously or with at least a temporal overlap.

After the needle holder has been retracted into the barrel and/or after the medicament container has been moved to the proximal replacement position there is provided access to the medicament container. For this, a control of the injection device may provide access to the medicament container. For this, a control may be configured to open or to release a closure providing access to the medicament container located inside the housing of the injection device. Alternatively or additionally, the control of the injection device may conduct an automated injection procedure. Here, the injection device may be configured to eject the medicament container, e.g. by opening or by releasing a closure formed as a lid. The closure may be pivoted into an open configuration and may be mechanically connected to the barrel. By pivoting or moving of the closure or lid also the barrel connected or mechanically coupled to the closure or lid may experience a respective pivoting motion thus exposing at least one end section of the barrel for gripping by the fingers of a user or patient of the injection device. Thereafter, the medicament container is removed from the housing and finally another medicament container, typically a filled medicament container is inserted into the housing.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(02)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(02)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM. The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present invention without departing from the scope of the invention. Further, it is to be noted, that any reference numerals used in the appended claims are not to be construed as limiting the scope of the invention.

### BRIEF DESCRIPTION OF THE FIGURES

In the following, numerous examples of the medicament container and of an injection device will be described in greater detail by making reference to the drawings, in which:

FIG. 1 is a schematic side view of an injection device equipped with a container, wherein the container is in the proximal replacement position,

FIG. 2 shows the injection device according to FIG. 1 with the medicament container in the distal injection position,

FIG. 3 shows the device according to FIGS. 1 and 2 after emptying of the container,

FIG. 4 shows the injection device according to FIGS. 1-3 with the container in distal injection position and after retracting the bung and the needle holder of the container into the barrel of the container, and

FIG. 5 shows the injection device of FIG. 4 after returning of the container back into the proximal replacement position for exchanging the container by another container,

FIG. 6 is a schematic illustration of one example of a container in the proximal replacement position,

FIG. 7 shows the container of FIG. 6 in the distal injection position before dispensing of the medicament,

FIG. 8 shows the container of FIG. 7 after expelling of the medicament with the injection needle retracted into the barrel of the container and with the container returned in the proximal replacement position and

FIG. 9 shows the configuration of the container of FIG. 8 with the plunger disconnected from the bung of the container,

FIG. 10 is a flowchart of a method of exchanging a medicament container,

FIG. 11a is an enlarged side view of the bung according to FIGS. 6-9,

FIG. 11b is a front view of the distal end face of the bung,

FIG. 12 is an enlarged view of a needle holder used with a container as illustrated in FIGS. 13-16,

FIG. 13 shows another example of a container located in a proximal replacement position,

FIG. 14 shows the container of FIG. 13 in the distal injection position,

FIG. 15 shows the container of FIG. 14 after connecting the bung with the needle holder and after retracting the bung and the needle holder into the barrel of the container,

FIG. 16 shows the container in the proximal replacement position before the plunger is disconnected from the bung, and

FIG. 17 is a cross-section through the barrel and the needle holder according to A-A of FIG. 14.

### DETAILED DESCRIPTION

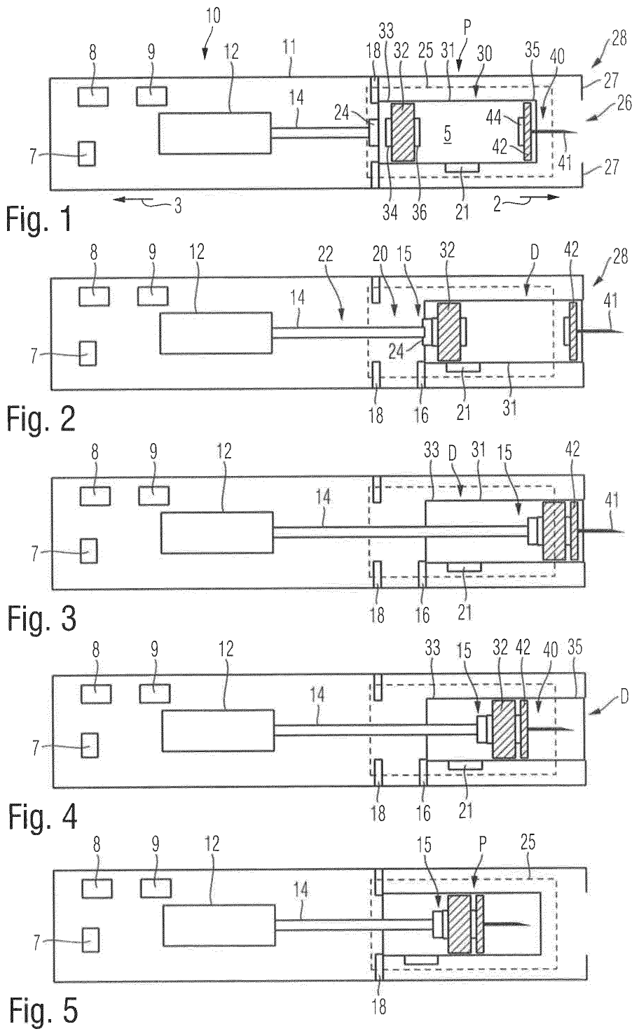

In the sequence of FIGS. 1-5 an injection device 10 is schematically illustrated. The injection device 10 is configured or implemented as an auto-injector that is capable to pierce the skin of a patient with an injection needle and to expel a predefined amount of a medicament, i.e. a dose of the medicament, into the pierced tissue. The injection device may comprise a handheld pen-type injector. When implemented as an auto-injector the injection device 10 is configured to advance the injection needle 41 relative to a housing 11 in the distal direction 2 so as to puncture the skin of the patient.

After completion of the injection procedure the injection device 10 is configured to retract and to withdraw the injection needle from the skin of the patient and to retract the needle inside the housing 11 of the injection device 10. The injection device 10 is configured to receive a container 30 having a tubular-shaped barrel 31 filled with a liquid medicament 5. The housing 11 comprises a proximal end and an oppositely located distal end 2. In FIG. 1, the proximal direction 3 and the distal direction 2 are indicated by respective arrows. The distal end 28 of the injection device 10 is the dispensing end facing towards the pierceable skin of the patient. The medicament container 30 comprises a piston or bung 32 sealing the tubular barrel 31 towards the proximal direction 3. The bung 32 is slidably arranged inside the barrel 31.

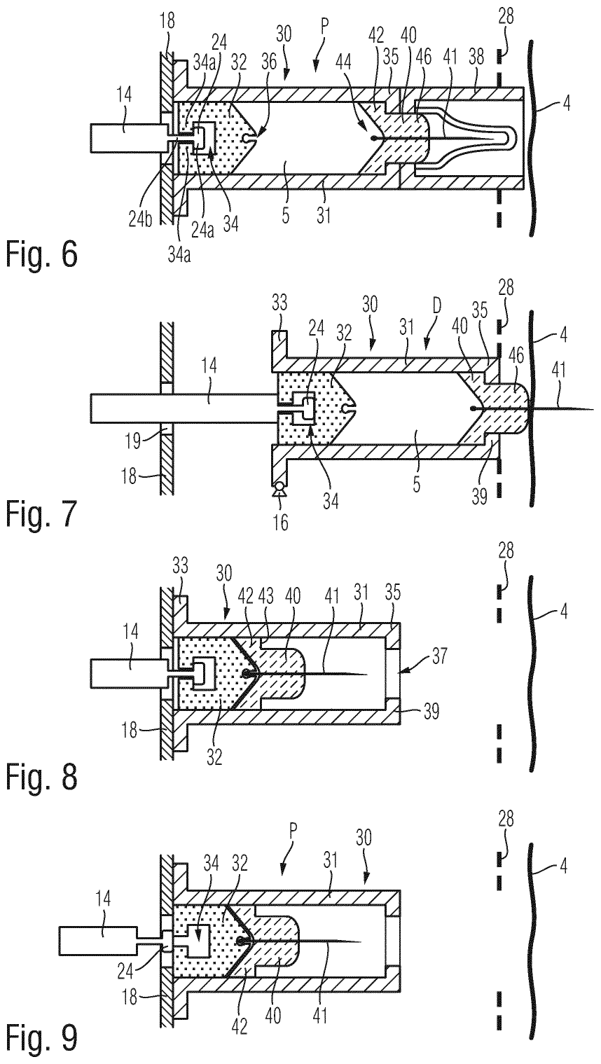

In an initial configuration as illustrated in FIG. 1 the bung 32 is located at or near a proximal end 33 of the barrel 31. At or near the distal end 35 of the barrel 31 there is provided a needle holder 40. The needle holder 40 is equipped with an injection needle 41. Optionally, the needle holder 40 comprises a seal 42 in fluid tight engagement with the inside facing sidewall of the barrel 31. The barrel 31 may be open ended at its distal end 35. It may comprise a radially inwardly extending flange 39 as illustrated in the more detailed illustration of FIGS. 6-9. The distal end of the injection needle 41 may protrude from the distal end 35 of the barrel 31 in the initial configuration as illustrated in FIG. 1. The needle holder 40 comprises a counterpart connector 44 complementary shaped to a connector 36 of the bung 32. The connector 36 of the bung 32 faces in distal direction 2, hence towards the needle holder 40. The counterpart connector 44 of the needle holder 40 faces towards the proximal direction 3, hence towards the bung 32.

The injection device 10 further comprises a drive mechanism 12 having at least a plunger 14 being moveable along the axial direction, hence towards the distal direction 2 and towards the proximal direction 3 relative to the housing 11. The drive mechanism 12 may be all mechanically implemented. It may be driven by a mechanical energy reservoir, such as a spring. With other examples the drive mechanism 12 may comprise an electromechanical drive and may be electrically controlled, e.g. by a control 7 of the injection device 10. Moreover, the injection device comprises a dose setting member 8 and a trigger 9. By means of the dose setting member 8 at least the size of a dose or other dosing parameters, such as the injection velocity may be modified or set. By means of the trigger a dose dispensing action can be initiated and/or controlled or prematurely interrupted if required.

The injection device further comprises a container moving mechanism 20 by way of which the container 30 can be moved relative to the housing 11 between a proximal replacement position P as illustrated in FIGS. 1 and 5 and a distal injection position D as illustrated in FIGS. 2-4. The container moving mechanism 20 may comprise a separate drive 21 configured to displace the container 30 relative to the housing 11. In addition to that or alternative to the drive 21 also the plunger 14 and the drive mechanism 12 may provide or form a driver 22 configured for moving the container 30 between the distal injection position D and the proximal replacement position P.

The plunger 14 comprises a connector 24 located at a distal end of the plunger 14 and hence facing towards a proximal end of the bung 32. The bung 32 comprises a counter connector 34 complementary or correspondingly-shaped to the connector 24 of the plunger 14. The counter connector 34 is located at a proximal side of the bung 32 and faces towards the proximal direction 3. The connector 24 and the complementary shaped counter connector 34 are configured to releasably connect to form a plunger bung assembly 15 as illustrated in FIG. 2. The mutually corresponding connector 24 and counter connector 34 may be configured to form a snap fit, a positive fit or a positive locking of the bung 32 and the plunger 14.

The mutual interconnection between the plunger 14 and the bung 32 is tension stable and exhibits a tensile strength that is larger than a friction force required for moving the bung 32 relative to the barrel 31. The interconnection between the plunger 14 and the bung 32 may be of load limiting type. If for instance a proximally directed movement of the bung 32 is impeded or blocked and if the plunger 14 exerts a tensile stress to the bung 32 exceeding the predefined maximum load the interconnection between the plunger 14 and the bung 32 abrogates. The container moving mechanism 20 may comprise a sliding mechanism to slidably support the medicament container 30 inside the housing 11.

In FIG. 1 an initial and undeployed configuration of the injection device 10 is schematically illustrated. The container 30 filled with the injectable medicament 5 has been inserted into the housing 11 and a protective cap 38 as illustrated in FIG. 6 has been removed prior to a first use of the injection device 10. After setting an initial dose and after an eventual priming procedure the medicament container 30 is displaced in distal direction 2 until it arrives at the distal injection position D. In the distal injection position D the distal end 35 of the container 30 may abut with an abutment section, e.g. in form of a flange 27 at or near a distal end 28 of the housing 11. When arriving at the distal injection position the injection needle 41 protrudes from the distal end 28 of the housing 11. Presuming that the distal end 28 of the housing 11 is in contact with an injection site of a patient the respective tissue, hence the skin 4 of the patient will be pierced as indicated in FIG. 7.

The distally directed motion and displacement of the medicament container 30 into the distal injection position D is governed or conducted by at least one of the drive mechanism 12 and a separate drive 21 of the container moving mechanism 20. The drive 21 is only optional. Generally, the drive mechanism 12 and the plunger 14 may be operable to displace the container 30 into the distal injection position. Here, the container moving mechanism 20 is constituted by the drive mechanism 12 and the plunger 14 connected to the bung 32. The plunger 14 can be driven in distal direction 2 until it gets in abutment with the bung 32. The friction force between the bung 32 and the inside surface of the tubular barrel 31 is typically larger than a force required for moving the container 30 from the proximal replacement position P towards and into the distal injection position D. The friction force between the bung 32 and the barrel 31 may be even larger than a force required for piercing the skin 4 by the needle 41.

In FIG. 3 a configuration is illustrated wherein the entirety of the medicament 5 has been dispensed through the injection needle 41. Generally, the content of the container 30 may be sufficient to conduct several injection procedures.

If a last dose of the medicament 5 has been dispensed the bung 32 reaches a distal most position inside the barrel 31 as illustrated in FIG. 3. There, the distally facing connector 36 of the bung 32 mechanically engages with the proximally facing counter connector 44 of the needle holder 40. In this way, a tension stable connection between the bung 32 and the needle holder 40 is established. Since the bung 32 is connected to the plunger 14 a movement of the plunger 14 towards the proximal direction 3 induced by the drive mechanism 12 leads to a respective displacement of the bung 32 and of the needle holder 40 provided that the medicament container 30 is secured inside the housing 11 against a proximally directed displacement or movement.

In order to prevent a premature proximally directed movement of the container 30 from the distal injection position D towards the proximal replacement position P the injection device 10 comprises a distal retainer 16. The distal retainer 16 is configured to get in axial abutment with at least a portion, e.g. with a proximally facing flange or with an end face of the barrel 31 of the medicament container 30. As illustrated in FIGS. 2-4 the distal retainer 16 is located at a longitudinal position of the housing 11 that coincides with the proximal end face of the barrel 31. In this way and when arriving at the distal injection position D the barrel 31 is axially confined between the flange 27 at the distal end of the housing 28 and the distal retainer 16.

The distal retainer 16 is at least temporally deactivatable to allow and to support a proximally directed movement or retraction of the container 30 relative to the housing 11 towards the proximal replacement position P. After termination of a last injection from the container 30 as indicated in FIG. 3, the distal retainer 16 is activated so as to prevent a proximally directed movement of the container 30 relative to the housing 11. A proximally directed movement of the plunger 14 connected to the bung 32 and connected to the needle holder 40 then leads to a retraction of the needle holder 40 and of the injection needle 41 into the interior of the barrel 31 until the distal end of the injection needle 41 is located proximal from the distal end 35 of the barrel 31. This situation is indicated in FIG. 4.

Thereafter, the distal retainer 16 is deactivated or released so as to allow and to support a proximally directed movement of the container 30 towards and into the proximal replacement position P. The housing 11 may comprise a closure 25, e.g. equipped with a lid providing access to the container 30 only when the container 30 is in the proximal replacement position P. The closure 25 may be limited in size and may be only slightly larger than the barrel 31 of the cartridge. In this way and as long as the needle 41 protrudes from the distal end 35 of the barrel 31 the container 30 may be hindered from removal of the housing 11.

The sequence of FIGS. 6-9 shows one example of the container 30 in more detail as it is positioned inside the housing 11 of the injection device 10. In the initial configuration as illustrated in FIG. 6 the distal end 35 of the container 30 is provided with a protective cap 38. In The sequence of FIGS. 6-9 there are always provided vertical lines 28, 4. The line 28 represents the distal end of the housing 11 of the injection device 10 and the vertical line 4 represents the pierceable skin of a patient. In the initial configuration as illustrated in FIG. 6 a portion of the protective cap 38 protrudes from the distal end 28 of the housing. Typically, it protrudes through the orifice 26 as illustrated in FIG. 1. A user may grasp the protruding portion of the protective cap 38 and withdraw the protective cap thus exposing the injection needle 41. In the proximal replacement position P the distal end of the injection needle 41 is located entirely inside the housing 11. It does not yet protrude from the housing.

With the example of FIGS. 6-9 the needle holder 40 comprises a seal 42 in sealing and fluid tight engagement with an inside of the barrel 31. As illustrated in FIG. 8 the barrel 31 comprises a radially inwardly extending flange 39 at its distal end 35. The annular-shaped and radially inwardly extending flange 39 encloses a distal orifice 37 at the distal end face of the barrel 31. Through this orifice 37 there extends at least a portion of the needle holder 40. The needle holder 40 comprises a stepped down distally protruding socket section 46 protruding through the orifice 37 of the barrel 31 when in the initial configuration as illustrated in FIGS. 6 and 7. The injection needle 41 extends entirely through the needle holder 40. The seal 42 comprises a radially widening shoulder proximally adjacent to the stepped down socket section 46. The circumference or radial extension of the shoulder portion 46 is larger than the cross-section or inner diameter of the orifice 37. Hence, a distally facing abutment side of the shoulder portion 43 is in axial abutment with a proximally facing side of the flange 39.

The hollow and cannula type injection needle 41 comprises a distal tipped end and further comprises a counterpart connector 44 at its proximal end. The counterpart connector 44 may comprise a bulged shape to interconnect with the connector 36 located at the distal side of the bung 32. As illustrated in FIGS. 11a and 11b the connector 36 comprises a receptacle 34b configured to receive the bulge of the injection needle 41. As illustrated in FIG. 11b the receptacle 34b is formed in a tapered radial central portion of the bung 32. The receptacle 34b is intersected by two perpendicularly oriented slits thus forming four lobes or wings 36a, 36b, 36c and 36d being resiliently deformable so as to releasably engage with the bulge of the counterpart connector 44 of the injection needle 41.

As illustrated further in FIG. 11a the bung 32 comprises a counterpart connector 34 at a proximal end and facing towards the plunger 14. The counterpart connector 34 is configured to engage with a T-shaped connector 24 of the plunger 14. For this the connector 24 comprises a radially widened head 24a at a distal end connected with a proximal portion of the plunger 14 via a neck portion 24b. The neck portion 24b is configured to extend through a through opening formed by and extending between oppositely located radially inwardly extending flange portions 34a at the proximal end of the bung 32. The bung 32 is made of a resilient material and the flange portion 34 may be configured to widen radially as the head 24a of the plunger is urged into the receptacle 34b of the counterpart connector.

As illustrated in FIGS. 6-9 the connector 24 and the counterpart connector 34 are configured to form a snap fit connection that establishes automatically as the plunger 14 is driven in distal direction 2 against the bung 32. As the bung 32 has reached a distal end position, which is currently not illustrated and as the connector 36 mechanically connects to the counterpart connector 44 the bung 32 is in a tension stable connection with the injection needle 41 and the needle holder 40. Then, the needle holder 40 together with the bung 32 can be retracted into the barrel 31 and the container 30 can be retracted from the distal injection position D to and into the proximal replacement position P as illustrated in FIGS. 8 and 9.

Once arrived at the proximal replacement position P as illustrated in FIG. 8 the proximal end 33 of the container 30 is in axial abutment with a proximal retainer 18. The proximal retainer 18 may be connected to or may be integrally formed with the housing 11. The proximal retainer 11 comprises a through opening 19 that is large enough for the plunger 14 to pass through. The through opening 19 is in fact smaller than the outer dimensions of the proximal end 33 of the barrel 31 as well as the outer dimensions of the bung 32.

When arriving in the proximal replacement position P as illustrated in FIG. 8 the proximal end 33 of the barrel 31 and a proximal end of the bung 32 are in axially abutment with the proximal retainer 18. In this way, any further proximally directed displacement of the container 31 and of its bung 32 is impeded and blocked by the proximal retainer 18. Since the mutual interconnection between the plunger 14 and the bung 32 is of load limiting type the interconnection between the connector 24 and the counterpart connector 34 can be abrogated as the plunger 14 is moved further in proximal direction so that a mechanical tension across the interface of plunger 14 and bung 32 exceeds the predefined maximum load.

After applying such excessive force to the plunger 14 in proximal direction 3 the plunger 14 disconnects from the bung 32 and hence from the container 30. The container 30 can then be removed or replaced by another container.

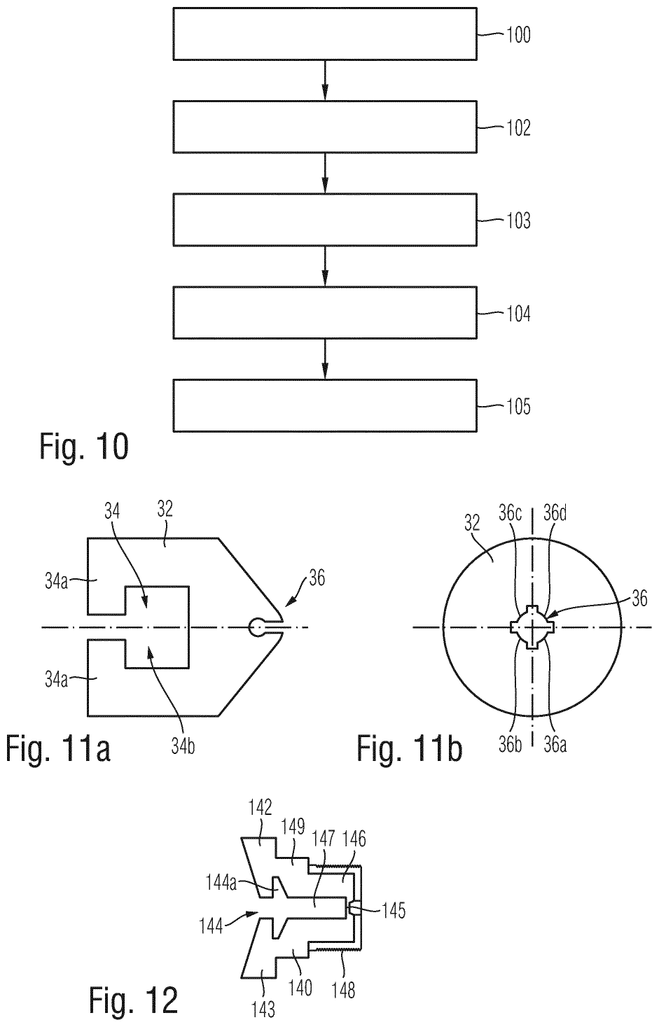

In FIG. 10 various steps of conducting a method of exchanging a medicament container are illustrated. The method starts after completion of an injection procedure. In a first step 100 a needle holder 40 is retracted into a barrel 31 of a medicament container 30. In a further step 102 the medicament container 30 is moved relative to a housing 11 of the injection device 10 from a distal injection position D into or towards a proximal replacement position P.

The sequence of the steps 100 and 102 may also be exchanged or the steps 100 and 102 may be conducted at a certain temporal overlap. The steps 100 and 102 may even be conducted simultaneously. With either step 100 or 102 the injection needle 41 still penetrating the skin 4 of a patient after completion of the injection procedure is withdrawn from the skin 4 of the patient.

For the method it is generally sufficient when only step 100 is performed so that the needle holder 40 is retracted into the barrel 31 of the medicament container 30. Step 102 may be only optional. After the needle holder 40 has been retracted into the barrel 31 in step 103 there is provided access to the medicament container 30 which is still located inside the housing 11 of the injection device 10. In a subsequent step 104 the medicament container 30 is removed from the housing 11 and in the further step 105 another medicament container 30 is inserted into the housing 11 of the injection device 10. Thereafter, subsequent dose setting and injection procedures may take place with the new medicament container.

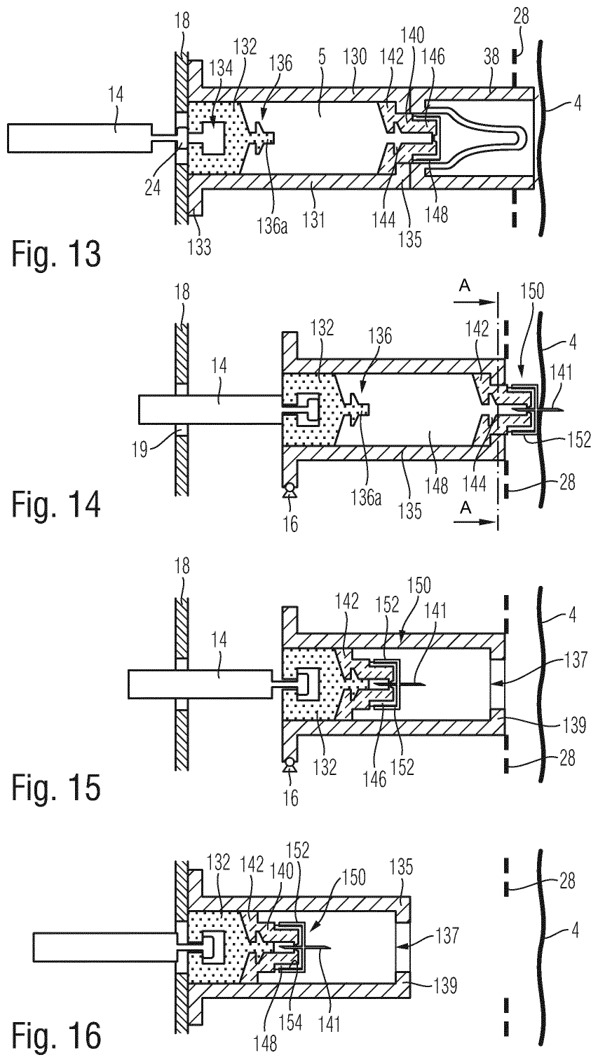

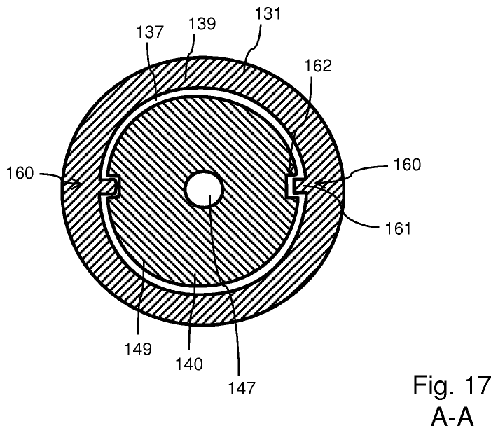

In FIG. 12 a further example of a needle holder 140 is illustrated. The needle holder 140 is also insertable into the interior of a barrel 131 of a medicament container 130 as illustrated in more detail in FIGS. 13-16. The illustration of FIGS. 13-16 is in close analogy to the illustration of FIGS. 6-9. Here, identical or similar components are indicated with identical or similar reference numbers as used before in connection with FIGS. 6-9. Similar components are indicated with reference numbers increased by 100.

The needle holder 140 comprises a step down socket portion 146 at its distal end protruding through an orifice 137 at a distal end 135 of a barrel 131 of the medicament container 130. The orifice 137 and the distal end 135 are laterally confined by a radially inwardly extending flange 139 protruding radially inwardly from a sidewall of the barrel 131. In the initial configuration as illustrated in FIG. 13 the socket 146 protrudes distally from the distal end of the barrel 135. The socket 146 is a threaded socket comprising an outer thread 148. The outer thread 148 is configured to threadedly engage with an inner thread 154 of a cupped receptacle 152 of a needle assembly 150 illustrated in FIG. 14. The step of attaching of the needle assembly 150 to the socket 146 of the needle holder 140 is not illustrated.

In FIG. 14 the needle assembly 150 is illustrated as the injection needle 141 already pierces the skin 4 of the patient. The needle assembly 150 comprising the cupped receptacle 152 intersected by the double-tipped injection needle 141 may be readily assembled on the threaded socket 148 even before the protective cap 38 is taken away. The protruding socket 146 comprises a pierceable closure 145 that hermetically separates a hollow channel section 147 of the needle holder 140 from an outer surface of the needle holder 140. The needle holder 140 is made of an elastomeric material, such as natural or synthetic rubber. The pierceable closure 145 forms a kind of a pierceable septum located in a radial center of the threaded socket 146.

The needle holder 140 comprises a radially widened shoulder section 143 proximally adjacent to the threaded socket 146. The needle holder 140 further comprises an axial mid-section 149 located axially between the shoulder portion 143 and the threaded socket 146. The cross-section or diameter of the mid-section 149 matches with the inner diameter of the orifice 137 of the barrel 131. The outer circumference of the seal 142 and hence of the shoulder section 143 matches with the inner circumference or inner cross-section of the sidewall of the barrel 131 and the threaded socket 146 is reduced in diameter compared to the inner cross-section of the orifice 137. The diameter reduction of the threaded socket 146 compared to the mid-section 149 matches with the radial thickness of the threaded engagement of the threaded socket 146 with the cupped receptacle 152.

In this way, the needle assembly 150 in threaded engagement with the threaded socket 146 can be retracted together with the needle holder 140 in proximal direction 3 into the barrel 131 of the container 130.

The channel section 147 comprises a snap feature thus forming a counterpart connector 144 to engage with a connector 136 of the bung 132. The bung 132 comprises a counterpart connector 134 that is substantially identical to the counterpart connector 34 as illustrated and described in connection with FIGS. 6-9. The connector 136 comprises a snap feature 136a to engage with the snap feature 144a of the counterpart connector 144. Here, the snap feature 136a is a male snap feature and the snap feature 144a is a female snap feature extending into or spatially overlapping with the channel section 147 through which the medicament 5 is to be expelled once the double-tipped injection needle 141 has pierced the pierceable closure 145 at the distal end face of the needle holder 140.

The mutually corresponding snap features 136a, 144a comprise beveled structures facing in distal direction 2 so as to facilitate establishing of a mechanical connection between the bung 132 and the needle holder 140. A withdrawal force for disconnecting the bung 132 and the needle holder 140 is therefore substantially larger than a force required for establishing the snap fit engagement of the connector 136 and the counterpart connector 144.

In FIG. 17 a cross-section A-A according to FIG. 14 is illustrated. FIG. 17 is illustrative of a rotation lock 160 configured to prevent a rotation of the needle holder 140 relative to the barrel 131 or relative to the container 130 as long as the needle holder 140 is located at the distal end 135 of the container 130. The rotation lock 160 comprises a radial protrusion 161 and a complementary shaped radial recess 162.

The radial protrusion 161 is located on one of the barrel 131 and the needle holder 140, wherein the complementary shaped radial recess 162 is located on the other one of the barrel 131 and the needle holder 140. In the illustration of FIG. 17 the protrusion 161 is located on the radially inwardly extending flange 139 at the distal end 135 of the barrel 131. The protrusion 161 protrudes radially inwardly into the orifice 137. The complementary shaped recess 162 is provided on an outer surface of the midsection 149 of the needle holder 140. In the initial configuration and hence prior to a retraction of the needle holder 140 into the interior of the container 130 or barrel 131 the midsection 149 axially or longitudinally intersects the orifice 137 radially confined by the flange 139.

As long as the at least one protrusion 161 and the at least one recess 162 are in mechanical engagement the needle holder 140 is rotationally fixed to the barrel 131. A user may then attach a needle assembly 150 to the threaded socket 146 protruding from the distal end 135 of the barrel 131. The rotational interlock between the barrel 131 and the needle holder 140 is of particular benefit to enable a screwing connection between the needle assembly 150 and the needle holder 140.

As illustrated in FIG. 17 there is provided a pair of substantially equally shaped rotation locks 160 circumferentially distributed around the outer circumference of the needle holder 140. With numerous rotational locks 160 a holding torque between the barrel 131 and the needle holder 140 can be equally distributed among the number of available rotational locks. Mechanical point loads to each rotation lock 160 can be thus reduced.

### LIST OF REFERENCE NUMBERS

2 distal direction
3 proximal direction
4 skin
5 medicament
7 control
8 dose setting member
9 trigger
10 injection device
11 housing
12 drive mechanism
14 plunger
15 plunger bung assembly
16 retainer
18 retainer
19 through opening
20 container moving mechanism
21 drive
22 drive
24 connector
24a head portion
24b neck portion
25 closure
26 orifice
27 flange
28 distal end
30 container
31 barrel
32 bung
33 proximal end
34 counterpart connector
34a flange portion
34b receptacle
35 distal end
36 connector
36a, b, c, d wings
37 orifice
38 cap
39 flange
40 needle holder
41 injection needle
42 seal
43 shoulder portion
44 counterpart connector
46 socket
130 container
131 barrel
132 bung
133 proximal end
134 counterpart connector
135 distal end
136 connector
136a snap feature
137 orifice
139 flange
140 needle holder
141 injection needle
142 seal
143 shoulder portion
144 counterpart connector
145 pierceable closure
146 threaded socket
147 channel section
148 outer thread
149 mid-section
150 needle assembly
152 receptacle
154 inner thread
160 rotational lock
161 protrusion
162 recess

1-15. (canceled)

16. An injection device for injecting of a dose of a liquid medicament, the injection device comprising:
a housing configured to receive a container, wherein the container comprising a tubular barrel filled with the liquid medicament is sealed by a bung in a proximal direction, and wherein the bung is slidably arranged inside the tubular barrel;
a drive mechanism having a plunger configured to urge against the bung in a distal direction to expel the dose of the liquid medicament from the container, wherein the plunger is configured to releasably connect to the bung to form a plunger bung assembly;
a container moving mechanism configured to move the container relative to the housing between a proximal replacement position and a distal injection position; and
at least one retainer configured to prevent a proximally-directed movement of the container relative to the housing,
wherein the drive mechanism is configured to retract the plunger and the plunger bung assembly along the proximal direction relative to the tubular barrel of the container.

17. The injection device according to claim 16, wherein the at least one retainer comprises a distal retainer configured to retain the container in the distal injection position.

18. The injection device according to claim 17, wherein the distal retainer is de-activatable to allow the proximally-directed movement of the container relative to the housing towards the proximal replacement position.

19. The injection device according to claim 16, wherein the at least one retainer comprises a proximal retainer configured to retain the container in the proximal replacement position.

20. The injection device according to claim 16, wherein the plunger comprises a connector to releasably connect with a counterpart connector of the bung.

21. The injection device according to claim 16, wherein the container moving mechanism is configured to slidably displace the container between the proximal replacement position and the distal injection position, and wherein the container moving mechanism comprises a drive to induce or to control a motion of the container relative to the housing.

22. The injection device according to claim 21, wherein the drive is provided by the plunger connected to the bung and movable relative to the housing.

23. The injection device according to claim 16, further comprising the container arranged inside the housing.

24. The injection device according to claim 23, wherein the container comprises a needle holder configured to hold an injection needle, and wherein the needle holder is retractable inside the tubular barrel.

25. The injection device according to claim 24, wherein the bung comprises a connector facing towards the needle holder, wherein at least one of the needle holder or the injection needle comprises a counterpart connector facing towards the bung, and wherein the connector and the counterpart connector are configured to form a mechanical interconnection between the bung and the needle holder.

26. The injection device according to claim 24, wherein the needle holder comprises a seal in fluid tight engagement with the tubular barrel of the container.

27. A container for a liquid medicament, the container comprising:
a tubular barrel;
a bung sealing a proximal end of the tubular barrel, wherein the bung comprises a connector facing towards a distal end of the tubular barrel; and
a needle holder configured to hold an injection needle and retractable inside the tubular barrel, wherein the needle holder comprises a seal in fluid tight engagement with the tubular barrel,
wherein at least one of the needle holder or the injection needle comprises a counterpart connector configured to engage with the connector to form a mechanical interconnection with the bung.

28. The container according to claim 27, wherein the needle holder comprises a socket configured to protrude axially through an orifice at a distal end of the tubular barrel, and wherein the socket comprises an outer thread to engage with an inner thread of a cup-shaped needle assembly.

29. The container according to claim 28, wherein the needle holder comprises a radially widening shoulder portion axially adjacent to the socket.

30. The container according to claim 27, further comprising a rotation lock configured to prevent a rotation between the needle holder and the tubular barrel, wherein the rotation lock comprises at least one radial protrusion configured to engage with at least one complementary-shaped radial recess.

31. The container according to claim 30, wherein the at least one radial protrusion is located on one of the needle holder and the tubular barrel, and the at least one complementary-shaped radial recess is located on the other of the needle holder and the tubular barrel.

32. The container according to claim 30, wherein the at least one radial protrusion faces inwardly from an inwardly facing circumference of a flange confining an orifice of the tubular barrel, and the at least one complementary-shaped radial recess is located on an outer surface of the needle holder.

33. The container according to claim 30, wherein the at least one radial protrusion and the at least one complementary-shaped radial recess extend in a longitudinal direction.

34. A method of exchanging a medicament container in an injection device, the method comprising:
retracting a needle holder into a barrel of the medicament container;
moving the medicament container relative to a housing of the injection device from a distal injection position into or towards a proximal replacement position;
providing access to the medicament container after the needle holder and/or after the medicament container has been moved to the proximal replacement position; and
removing the medicament container from the housing and inserting another medicament container into the housing.

35. The method according to claim 34, wherein moving the medicament container relative to the housing of the injection device from the distal injection position into or towards the proximal replacement position comprises moving the medicament container relative to the housing towards and into the proximal replacement position while the needle holder remains stationary relative to the barrel of the medicament container.
